(19) United States
(12) Patent Application Publication
(10) Pub. No.: US 2008/0075628 A1
(43) Pub. Date: Mar. 27, 2008

(54) STERILIZED MINOCYCLINE AND RIFAMPIN-CONTAINING MEDICAL DEVICE

(75) Inventors: Dianne Judd, Minneapolis, MN (US); Jennifer Raeder-Devens, St. Paul, MN (US); William Ferris, Minneapolis, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 11/535,762

(22) Filed: Sep. 27, 2006

(51) Int. Cl. A61L 2/00

(52) U.S. Cl. 422/22; 424/423

(57) ABSTRACT

A sterilized medical device comprising minocycline and rifampin is described. The device is setrerilized by e-beam raditation such that degradation of minocycline and rifampin is minimized. Methods for sterilizing minocycline and rifampin-containing devices by e-beam radiation and methods of manufacturing devices, which methods include e-beam sterilization, are also described.

### FIELD

This application relates to medical devices and methods of sterilizing medical devices; particularly drug-containing devices and more particularly minocycline and rifampin-containing devices.

### BACKGROUND

Implantable medical devices increasingly incorporate drugs to improve the performance of the medical device or reduce side effects associated with implantation of the device. Sterilization processes suitable for medical devices that do not incorporate drugs may not be suitable for such devices that incorporate drugs, due to incompatibilities of the sterilization process and the drugs. Examples of sterilization processes include steam sterilization (e.g., autoclaving), chemical sterilization (e.g., ethylene oxide or vaporized hydrogen peroxide), and sterilization via radiation (e.g., gamma or e-beam). Steam sterilization may not be compatible with drugs that degrade under high temperature or humidity conditions. Chemical sterilization may not be compatible with drugs that have chemical groups that react with the sterilization chemical, such as ethylene oxide. Radiation typically alters the chemical structures of drugs incorporated into medical devices. Accordingly, some drugs may be incompatible with radiation sterilization and may be more or less sensitive to gamma versus e-beam radiation, depending on the nature of the specific drug.

With regard to radiation sterilization, gamma radiation is capable of penetrating much further into a product or packaging than e-beam radiation because gamma radiation is higher energy radiation than e-beam. As such, gamma radiation may be preferred to e-beam in certain situations where the product is thick or dense and sterility throughout must be demonstrated. However, due to the set up of typical sterilization facilities, products sterilized by gamma radiation may be over sterilized or exposed to gamma radiation for a longer time than is needed to achieve sufficient sterilization. On the other hand, facilities for e-beam sterilization are typically more capable of limiting exposure of a product to the amount of radiation energy necessary to achieve sufficient sterilization. Accordingly, e-beam sterilization can be gentler than gamma radiation to products and their packaging. Regardless of the sterilization process employed, the compatibility of a drug or device with a particular sterilization process should be drug or device dependent if the sterilization level of the process is roughly equivalent.

The compatibility of a drug with a sterilization process is likely to vary from drug or device to drug or device and sterilization process to sterilization process. As such, selection of a sterilization process for a device incorporating a drug should be carefully considered. For example, ethylene oxide and radiation can lead to degradation of ketorloac tromethamine in medical devices incorporating ketorolac tromethamine. However, such degradation of ketorolac tromethamine may be held to acceptable levels using sterilization procedures that employ relatively low energy radiation, such as e-beam radiation, and minimum exposure to moisture, oxygen and light. Devices containing other drugs may require similar or different sterilization procedures.

For example minocycline, an antibiotic commonly employed in medical devices to reduce infection associated with use or implantation of the devices, degrades to unacceptable levels under steam sterilization, but does not appear to substantially degrade with ethylene oxide sterilization.

Rifampin, another antimicrobial agent that is employed in currently available implantable medical devices, is often used in combination with minocycline in medical devices to further reduce the risk of infection. Many of these rifampin-containing implantable medical devices are sterilized via treatment with ethylene oxide. In addition, numerous studies that report no detectable degradation or no loss in antimicrobial activity when devices containing rifampin are treated with ethylene oxide.

Sterilization procedures for medical devices should be carefully selected, especially in devices that incorporate drugs. If a device incorporates more than one drug, the selection of a sterilization process can be more difficult if the drugs are incompatible with different sterilization processes. For example, devices incorporating both minocycline and rifampin should not be steam sterilized because minocycline degrades under steam sterilization conditions. Commercially available medical devices, such as the Cook SPECTRUM® catheter and AMS 700™ Series penile implants available from American Medical Systems, incorporating a combination of rifampin and minocycline are currently sterilized with ethylene oxide. There have been reports of sterilizing catheters or cerebroventricular shunts containing both rifampin and minocycline or rifampin with gamma radiation. While such sterilization methods may be acceptable, rigorous study of the effects of sterilization processes on medical devices containing rifampin and minocycline is warranted such that better processes for such devices are obtained.

### BRIEF SUMMARY

A method for sterilizing a medical device comprising minocycline and rifampin is described. The method comprises sterilizing the device or a minocycline and rifampin-containing portion thereof with e-beam radiation. After sterilization with e-beam radiation, about 90% or more of the minocycline and 90% or more of the rifampin are recoverable from the device, relative to the amount recoverable prior to sterilization.

A method for manufacturing a medical device comprising minocycline and rifampin is described. The method comprises incorporating minocycline and rifampin in, on or about at least a portion of a medical device and sterilizing the at least a portion of the device with e-beam radiation. After sterilization with e-beam radiation, about 90% or more of the minocycline and 90% or more of the rifampin are recoverable from the device, relative to the amount recoverable prior to sterilization.

A sterilized medical device comprising minocycline and rifampin is described. The device comprises a polymeric material incorporating minocycline and rifampin. The polymeric material comprising minocycline and rifampin is sterilized by e-beam radiation such that the amount of minocycline and rifampin recoverable from the polymeric material after e-beam sterilization is 90% or more of the amount of minocycline and rifampin recoverable from the polymeric material prior to e-beam sterilization. The polymeric material may be disposed on or about a body member of the device. The body member may for the housing of a neurostimulator, an infusion device, a defibrillator, a pacemaker, a stent, a catheter, a lead, or the like.

Due to rigorous study of the effects of sterilization as disclosed herein, it has been surprisingly found that e-beam sterilization results in relatively high recovery of both rifampin and minocycline. This is not the case when the same drug-containing device is exposed to comparable levels of sterilization with ethylene oxide (EtO) or gamma radiation.

Subjecting implantable medical devices incorporating drugs to sterilization procedures that maximizes recovery of the drugs provides several advantages. For example, whether the chemical species of one or more degradants is known, degradants can pose a safety concern, especially with chronically implanted devices. In addition, minimizing degradation of a drug generally results in enhanced efficacy. These and other advantages will be readily understood from the following detailed descriptions when read in conjunction with the accompanying drawings.

### BRIEF DESCRIPTION OF THE DRAWINGS

FIG. 1 is a diagrammatic illustration of a cross-section of a device comprising anti-infective agent disposed within a coating layer of the device.

FIG. 2 is a diagrammatic illustration of a cross-section of a device comprising anti-infective agent disposed on the surface of a coating layer of the device.

FIG. 3 is a diagrammatic illustration of a cross-section of a device comprising anti-infective agent disposed in an intermediate layer and an outer layer of the device.

FIG. 4 is a diagrammatic illustration of a cross-section of a device comprising varying concentrations of anti-infective agent disposed in an intermediate layer and an outer layer of the device.

FIG. 5 is a diagrammatic illustration of a covering configured to receive an implantable medical device or portion thereof.

The figures are not necessarily to scale.

### DETAILED DESCRIPTION

It has been surprisingly shown that rifampin undergoes substantial degradation under ethylene oxide (EtO) sterilization conditions. The inventors have also found that e-beam sterilization of a medical device containing minocycline and rifampin results in enhanced recovery of rifampin as compared to ethylene oxide sterilization and enhanced recovery of minocycline and rifampin when compared to gamma radiation when the medical device is subjected to each of the sterilization processes to achieve comparable levels of sterilization.

As used herein, “or” means and/or unless otherwise specified.

Medical Device

Minocycline and rifampin may be incorporated in or on a medical device configured to release the minocycline or rifampin when implanted in a patient. For example, minocycline or rifampin may be embedded, coated, mixed, dissolved or dispersed on or in a vehicle. The vehicle may be disposed on, in or about at least a portion of the medical device. For example, the vehicle may be in the form of a coating or covering. In some embodiments, the vehicle may form the device; e.g., when the device is a catheter. Alternatively, the vehicle may be delivered proximate to a separate medical device as a therapeutic composition. A vehicle as described herein may take the form of a coating layer 25, 25′ as described in association with the figures presented herewith.

Minocycline and rifampin may be released from a device or vehicle at any rate sufficient to kill or inhibit growth of a microorganism. By “release” it is meant that minocycline or rifampin is located at a position such that minocycline or rifampin may contact a microorganism. In some circumstances, minocycline or rifampin will be considered “released” while still in contact with the vehicle. Preferably minocycline or rifampin is released for a duration sufficient to ward off a potential infection following implantation of a medical device.

The rate at which minocycline or rifampin 20 may be released from a vehicle or coating layer 25, 25′ into tissue may be controlled by properties of the vehicle or coating layers 25, as well as the manner in which minocycline or rifampin is disposed on or in the vehicle or coating layers 25. A further discussion of such details is provided below.

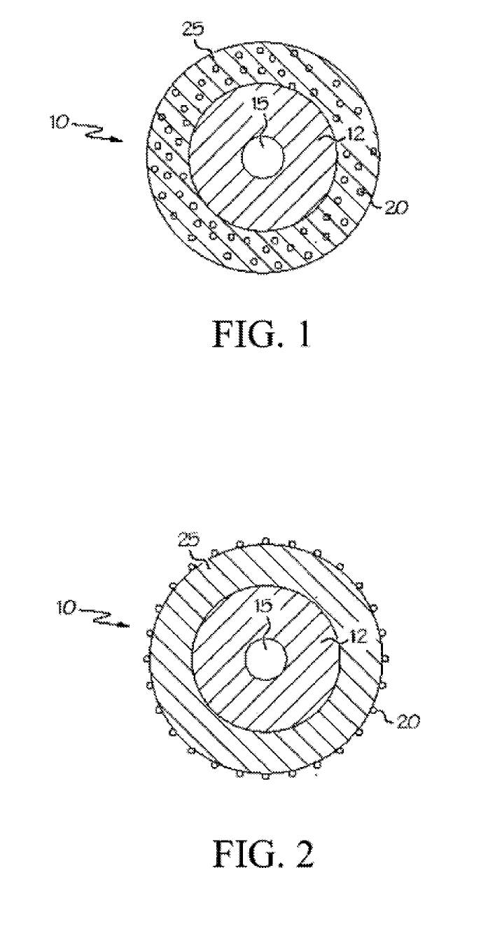

Various embodiments of the invention provide an implantable medical device 10 comprising a body member 12 into, onto, or about which minocycline or rifampin 20 is disposed. The medical device 10 may be any implantable medical device, such as a lead, a stent, a catheter, a neurostimulator such as an implantable pulse generator, a pacemaker, a defibrillator, an infusion device, and the like. Minocycline or rifampin 20 may be associated with the surface of the implantable medical device 10 in any fashion such that, after implanting the device 10, an infection may be prevented. For the sake of convenience, FIGS. 1-4 shown medical device 10 as a catheter comprising a lumen 15, but it should be understood that the discussion regarding these figures may be applicable to any implantable medical device 10, whether or not it comprises a lumen 15.

FIGS. 1-3 show examples of associations of minocycline and rifampin 20 with a surface of medical device 10. FIG. 1 shows that minocycline or rifampin 20 may be disposed in coating layer 25 disposed about a body member 12 of device 10. Coating layer 25 may be a vehicle, such as a polymeric vehicle. While FIG. 1 shows minocycline or rifampin 20 disposed throughout the coating layer 25, minocycline or rifampin 20 may be disposed within one or more portions of the coating layer 25 (not shown). FIG. 2 shows that minocycline or rifampin 20 may be disposed on the surface of the coating layer 25. If minocycline or rifampin 20 is disposed partially within the coating layer 25 or body member 12 or other layer and partially protrudes from a surface of the coating layer 25 or body member 12 or other layer, the minocycline or rifampin 20 is considered both disposed in and disposed on the coating layer 25 or body member 12 or other layer. Further, while not shown, it will be understood that minocycline or rifampin 20 may be both disposed in and disposed on the coating layer 25, body member 12, or other layer of the device 10.

It will be understood that agent 20 as depicted in FIGS. 1 and 2, other subsequent Figures, and throughout the present disclosure may refer to either minocycline or rifampin 20. That is, a given agent 20 depicted in FIG. 1A may be, e.g., rifampin, and a different agent 20 may be, e.g., minocycline.

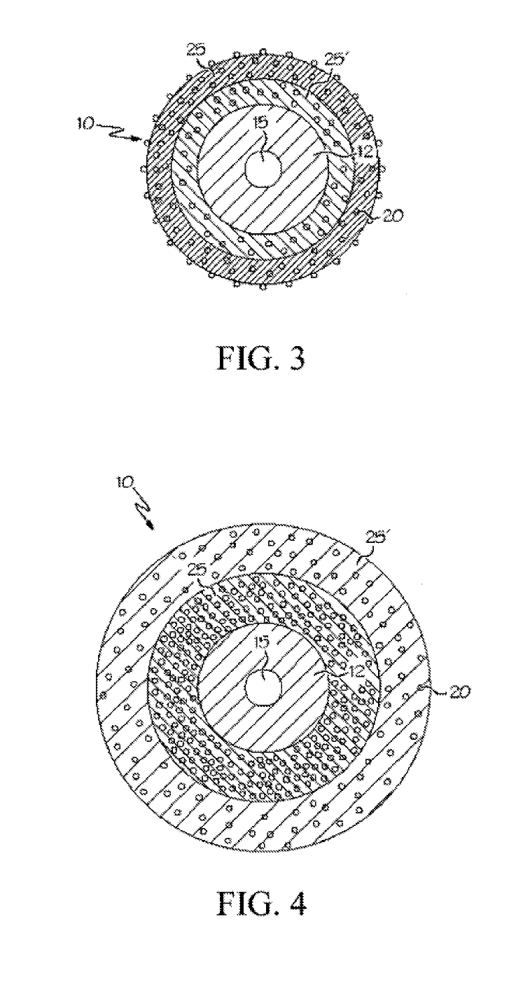

In various embodiments, minocycline or rifampin 20 are disposed on or in more than one layer of device 10. For example, minocycline or rifampin 20 may be disposed on or in a body member 12 of device 10 (not depicted) or on or in one or more coating layer 25 of device 10. FIG. 3 shows an embodiment where one of minocycline or rifampin 20 is disposed on or in a first coating layer 25, and the other of minocycline or rifampin 20 is disposed on or in a second coating layer 25′. Of course, two, three, four, five, six, or more coating layers 25 may be disposed about body member 12 of device 10.

The concentration of minocycline or rifampin 20 within various layers (depicted as body member 12 or coating layer 25, 25′) may be the same or different. Any concentration may be used. For example, minocycline or rifampin 20 may comprise about 0.1% to about 50%, or from about 1% to about 10%, of the weight of the layer. In some circumstances, it may be desirable to place a higher concentration minocycline or rifampin 20 in one or more layers relative to other layers. For example, to obtain a substantially constant release rate of minocycline or rifampin 20 over time it may be desirable for an underlying layer 25 to have a higher concentration of minocycline or rifampin and less in an overlying layer 25′. FIG. 4 shows a device 10, where first coating layer 25 comprises a higher concentration of minocycline or rifampin 20 within or on intermediate coating layer 25 than in outer coating layer 25′ or body member 12. Minocycline or rifampin 20 may elute out of outer coating layer 25′ into body tissue. Increased initial concentration of minocycline or rifampin 20 in intermediate coating layer 25 may effectively replenish the supply of anti-infective agent 20 in outer coating layer 25′ such that minocycline or rifampin 20 may continue to elute into tissue as substantially the same concentration over time. Of course other variations are possible. For example, it may be desirable to place a faster eluting agent, such as minocycline, 20 in an intermediate layer 25 and a slower eluting agent, such as rifampin, 20 in an outer layer 25′ such that minocycline and rifampin 20 reach the tissue at substantially simultaneously over a period of time.

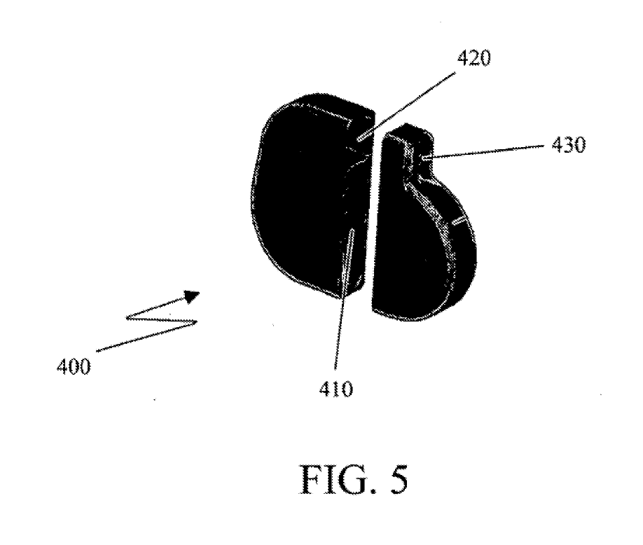

FIG. 5 shows an exemplary embodiment of a covering 400 configured to be disposed about an implantable medical device, such as a neurostimulator, a pacemaker, a drug infusion device, and the like. The covering may comprise one or more coating layers 25, 25′ (not shown) into or onto which minocycline or rifampin 20 (not shown) may be disposed. The covering 400 depicted in FIG. 5 comprises an opening 410 configured to snuggly receive an implantable medical device. The covering 400 may also comprise an opening 420 for a header of the device and an opening 430 for an accessory device, such as a lead, a lead extension, or a catheter. It will be understood that covering may be in any form and may be configured to be disposed about any implantable medical device. For example, covering may be a boot, a jacket, a sleeve, a sheath, and the like.

Coating layers 25, 25′ may comprise polymeric materials designed to control the rate at which rifampin or minocycline 20 is released, leached, or diffused from the polymeric material. As used herein, “release”, “leach”, “diffuse”, “elute” and the like are used interchangeably when referring to rifampin or minocycline 20 with respect to a vehicle, coating layer 25 or body member 12 of a delivery element. Any known or developed technology may be used to control the release rate. For example, a coating layer may be designed according to the teachings of WO/04026361, entitled “Controllable Drug Releasing Gradient Coating for Medical Devices.”

Coating layer 25 of device 10 may be in the form of a tube, sheath, sleeve, coating, or the like. Coating layer 25 may be extruded, molded, coated on body member 12, grafted onto body member 12, embedded within body member 12, adsorbed to body member 12, etc. Polymers of coating layers 25 may be porous or non-porous. Porous materials known in the art include those disclosed in U.S. Pat. No. 5,609,629 and U.S. Pat. No. 5,591,227. Typically polymers are non-porous. However, non-porous polymers may be made porous through known or developed techniques, such as extruding with CO₂ or by foaming the polymeric material prior to extrusion or coating.

Examples of suitable polymeric materials that may be used to form one or more coating layer 25 include bioerodable or biostable polymeric materials. Suitable bioerodable polymers include synthetic or natural bioabsorbable polymers. As used herein, “bioerodable”, “”biodegradable”, “bioabsorbable”, and the like are used interchangeably. Such polymers are recognizable and identifiable by one of ordinary skill in the art. Non-limiting examples of synthetic, biodegradable polymers include: poly(amides) such as poly(amino acids) and poly(peptides); poly(esters) such as poly(lactic acid), poly(glycolic acid), poly(lactic-co-glycolic acid), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), fibrin, fibrinogen, cellulose, starch, collagen, and hyaluronic acid, copolymers and mixtures thereof. The properties and release profiles of these and other suitable polymers are known or readily identifiable. It will be understood that minocycline or rifampin may elute from an intact vehicle or may be released upon degradation of the vehicle. In some embodiments, the biodegradable vehicle is a microcapsule. In another embodiment, the bioerodable vehicle is in the form of a gauze or wrap.

Suitable biostable materials include organic polymers such as silicones, polyamines, polystyrene, polyurethane, acrylates, polysilanes, polysulfone, methoxysilanes, and the like. Other polymers that may be utilized include polyolefins, polyisobutylene and ethylene-alphaolefin copolymers; acrylic polymers and copolymers, ethylene-covinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; carboxymethyl cellulose; polyphenyleneoxide; and polytetrafluoroethylene (PTFE). In various embodiments of the invention, the biostable vehicle comprises silicone or polyurethane.

In some embodiments, the polymeric material may be a hydrogel. Any hydrogel suitable for use in a human may be used. Hydrogels are known and recognizable by those of skill in the art. In some embodiments, the hydrogel may be a polyvinyl pyrrolidone (PVP) hydrogel.

Depending upon the type of materials used to form coating layers 25, the coatings can be applied to the surface of a body member 12 or underlying coating layer 25 through any coating processes known or developed in the art. One method includes directly bonding the coating material to a surface of body member 12 or underlying coating layer 25. By directly attaching a polymer coating to the body member 12 or underlying coating layer 25, covalent chemical bonding techniques may be utilized. Body member 12 or underlying coating layer 25 surface may possess chemical functional groups on its surface such as carbonyl groups, primary amines, hydroxyl groups, or silane groups which will form strong, chemical bonds with similar groups on polymeric coating material utilized. In the absence of such chemical forming functional group, known techniques may be utilized to activate the material's surface before coupling the biological compound. Surface activation is a process of generating, or producing, reactive chemical functional groups using chemical or physical techniques such as, but not limited to, ionization, heating, photochemical activation, oxidizing acids, sintering, physical vapor deposition, chemical vapor deposition, and etching with strong organic solvents. Alternatively, the coating layer 25 may be indirectly bound to body member 12 or underlying coating layer 25 through intermolecular attractions such as ionic or Van der Waals forces.

Minocycline or rifampin 20 may be incorporated into a coating layer 25 in a variety of ways. For example, minocycline or rifampin 20 can be covalently grafted to a polymer of the coating layer 25, either alone or with a surface graft polymer. Alternatively, minocycline or rifampin 20 may be coated onto the surface of the polymer either alone or intermixed with an overcoating polymer. Minocycline or rifampin 20 or may be physically blended with a polymer of a coating layer 25 as in a solid-solid solution. Minocycline or rifampin 20 may be impregnated into a polymer by swelling the polymer in a solution of the appropriate solvent. Any means of incorporating minocycline or rifampin 20 into or on a coating layer 25 may be used, provided that minocycline or rifampin 20 may be released, leached or diffuse from coating layer 25 on or after contact of device 10 with bodily fluid or tissue.

A polymer of a coating layer 25 and minocycline or rifampin 20 may be intimately mixed either by blending or using a solvent in which they are both soluble. This mixture can then be formed into the desired shape or coated onto an underlying structure of the medical device. One exemplary method includes adding one or more of minocycline or rifampin 20 to a solvated polymer to form an anti-infective agent/polymer solution. The agent/polymer solution can then be applied directly to the surface of body member 12 or underlying coating layer 25; for example, by either spraying or dip coating device 10. As the solvent dries or evaporates, the agent/polymer coating is deposited on body member 12. Furthermore, multiple applications can be used to ensure that the coating is generally uniform and a sufficient amount of agent has been applied to device 10.

Alternatively, an overcoating polymer, which may or may not be the same polymer that forms the primary polymer of body member 12 or underling coating layer 25, and minocycline or rifampin 20 are intimately mixed, either by blending or using a solvent in which they are both soluble, and coated onto body member 12 or underling coating layer 25. Any overcoating polymer may be used, as long as the polymer is able to bond (either chemically or physically) to the polymer of an underlying layer of delivery element 10.

In addition, a polymer of a coating layer 25 may be swelled with an appropriate solvent, allowing minocycline or rifampin 20 to impregnate the polymer.

Minocycline or rifampin 20 may also be covalently grafted onto a polymer of a coating layer 25. This can be done with or without a surface graft polymer. Surface grafting can be initiated by corona discharge, UV irradiation, and ionizing radiation. Alternatively, the ceric ion method, previously disclosed in U.S. Pat. No. 5,229,172, may be used to initiate surface grafting.

E-Beam Sterilization

“E-beam”, also known as “electron-beam” radiation, as used herein, refers to a form of ionizing radiation resulting from a concentrated, high current stream of electrons generated by accelerators that produce a beam of electrons. The beam can be pulsed or continuous.

Once a device 10 containing minocyclin and rifampin is manufactured or assembled, it is subjected to e-beam sterilization. Prior to e-beam sterilization device 10 may be placed in appropriate packaging for shipment so that the device 10 and its packaging may be sterilized. Any e-beam sterilization procedure may be used. Preferably, the e-beam sterilization procedure results in a product sufficiently sterile for implantation in a human. In some embodiments, a device 10 incorporating minocycline and rifampin is sterilized such that greater than about 90% of the incorporated minocycline or rifampin is recoverable after sterilization. In some embodiments, a device 10 incorporating minocycline and rifampin is sterilized such that greater than about 95% of the incorporated minocycline or rifampin is recoverable after sterilization. It will be understood that if the minocycline or rifampin degrades, it will not be recoverable. It will be further understood that minocycline or rifampin may not degrade, but nonetheless be unrecoverable. Such un-degraded, unrecoverable minocycline or rifampin may become so intimately associated with the polymeric material, e.g. covalently bound, that it is not able to be extracted, and thus is unrecoverable. Alternatively, with some sterilization processes; e.g. steam sterilization, therapeutic agent 20, 30 may leach out of first or second polymeric layers 25, 35, effectively reducing the amount of therapeutic agent 20, 30 that may be recovered from the polymeric layer 25, 35.

Any suitable procedure for recovering minocycline or rifampin may be employed. Typically minocycline or rifampin will be extracted from polymeric material and the extracted product will be subject to HPLC analysis. Examples of suitable solvents for extraction include ethanol, tetrahydrofuran (THF), THF/ethanol mixtures, chloroform, toluene, ethyl acetate, and the like. Of course, preferred solvents will depend on the drug (minocycline or rifampin) and polymeric material used.

Typically, e-beam accelerators are operated at between about 3 MeV and 12 MeV. Some e-beam accelerators are capable of varying the energy at which they operate. Products are typically placed on a conveyer belt and moved through the e-beam accelerator. E-beam sterilization systems may contain sensors that allow for control of the speed of the conveyer if the e-beam current changes during processing so that the dose of e-beam radiation is held constant. Additionally, some e-beam systems are capable of holding the product at low temperatures to reduce the initiation of side chemical reactions. Typical doses of radiation for high bioburden materials are in the range of 25 to 35 kGy. In an embodiment, devices 10 are e-beam sterilized at a dose ranging from about 25 to about 40 kGy. In an embodiment, devices 10 are e-beam sterilized at a dose of greater than about 20 kGy.

Products sterilized by e-beam radiation may not need to be subjected to sterility testing if the product is subject to the appropriate dose of e-beam radiation. Dosimeters may be used to measure the amount of radiation to which a product is exposed. For additional information, see the American National Standard, ANSI/AAMI/ISO 1137-1994.

Additional guidance on appropriate sterilization with e-beam radiation or other methods may be found in Sterilization of healthcare products—Requirements for validation and routine control—Radiation sterilization, AAMI/ISO 11137; Sterilization of healthcare products—Radiation Sterilization—Selection of a sterilization dose for single production batch, AAMI/ISO TIR No. 15844; and Sterilization of medical devices—Microbiological methods, Part 1: Estimation of population of microorganisms on products, AAMI/ISO 11737-1.

Thus, embodiments of the STERILIZED MINOCYCLINE AND RIFAMPIN-CONTAINING MEDICAL DEVICE are disclosed. One skilled in the art will appreciate that the methods, systems and devices described herein can be practiced with embodiments other than those disclosed. The disclosed embodiments are presented for purposes of illustration and not limitation.

### EXAMPLE

E-beam Sterilization Causes Little Degradation of Rifampin and Minocycline

Materials and Methods: Minocycline and rifampin were incorporated into discs, sheet stock and silicone boots and subjected to Steam, EtO, gamma radiation and e-beam sterilization as follows:

| Sterilization | |
|---|---|
| method | Process Details |
| Steam | TEMPERATURE: 250° F. |
| | PRESSURE: 20 psi |
| | TIME: 20 min (+15 min dry) |
| EtO | PREHEAT: Pre-vacuum Rate: 7–13 kPa/min; Pressure (leak check)14–20 pKa |
| | Leak check duration 13–17 min; Leak check vacuum loss < 4 kPa |
| | CONDITIONING: Time: 30–35 min; # of humidity passes 4; Cold spot temp |
| | 43–53° C.; Load probe temp 43–53° C.; Relative humidity 55–85%; Pressure |
| | prior to gas injection 8–14 kPa |
| | INJECTION: Gas temp 15–75° C. |
| | EXPOSURE: Time 72–78 min; Cold spot temp 45–55° C.; Load probe temp |
| | 45–55° C.; Relative humidity 55–85%; Pressure 55–75 kPa; Net weight of gas |
| | used 122–132 g; Temperature variance < 3; EtO concentration 700–1000 g/L |
| | POST VACUUM: Rate 1–2 kPa/min; End pressure 20–30 kPa |
| | AIR FLUSH: Time 25–1000 min |
| | AERATION: Temperature 44–55° C.; Time 8–26 min |
| E-Beam | Acsion: Calibrated at 20° C., using FWT dosimeters that are heat aged (on a |
| | single doe measurement the maximum uncertainty is estimated to be +/−6% |
| | at a 95% confidence level) Specified dose: 20–31.6 kGy Actual dose: 22.2–31.6 kGy |
| Gamma | Steris: Specified dose: 25–40 kGy Actual dose: 25.8–26.4 kGy |

Extraction Process—Boot

Each boot was cut into pieces (24) with scissors. The pieces were extracted together in a 60-125 ml jar w/top. All the pieces were placed in jar, and 4:1 THF/ethanol (50 mL for thick boots; 15 mL for thin boots) was added. The pieces were allowed to swell, and the drug is extracted for 1 hour (1^{st} extraction). The extract was then collected and transferred to a 25-100 mL volumetric flask. A second 4:1 THF/ethanol (50 mL for thick boots w/holes; 10 mL for thin boots with holes) extraction was added to the jar, and the transfer pipette was rinsed with this second extract and discarded. The pieces were allowed to swell, and the drug is extracted for 1 hour (2^{nd} extraction). The extract was then collected and transferred to the 25-100 mL volumetric flask containing the first extract. A small amount of extra solvent was needed to make up to volume due to solvent lost to evaporation and polymer swell. This amount also served as a final rinse of both the polymer and the transfer pipette.

Extraction Process—Sheet Stock

Discs was cut from the sheet stock with a die press. The discs were extracted separate in a 1 oz jar w/top. 4:1 THF/ethanol (5 mL) was added. The pieces were allowed to swell, and the drug is extracted for 1 hour (1^{st} extraction). The extract was then collected and transferred to a 10 mL volumetric flask. A second 4:1 THF/ethanol (5 mL) extraction was added to the jar, and the transfer pipette was rinsed with this second extract and discarded. The pieces were allowed to swell, and the drug is extracted for 1 hour. (2^{nd} extraction). The extract was then collected and transferred to the 10 mL volumetric flask containing the first extract. A small amount of extra solvent was needed to make up to 10 mL due to solvent lost to evaporation and polymer swell. This amount also served as a final rinse of both the polymer and the transfer pipette.

HPLC Method

After extraction with THF/ethanol 4:1, extract was transferred to an HPLC vial and injected directly. The following high performance liquid chromatography (HPLC) method was used:

1. Chromatographic Conditions:
Instrument: Agilent 1100 stack
Column: Prodigy analytical column (150×4.6 mm)
Detection mode: UV detection at 238 nm
Flow rate: 1.0 mL/min
Column temperature: 25° C.
Injection volume: 10 uL
Mobile phase A: 1.4 g/L dibasic sodium phosphate buffer pH 6.8
Mobile phase B: ACN 100%

2. Gradient

| Time (min). | % B |
|---|---|
| 0 | 12 |
| 10 | 36 |
| 25 | 75 |

3. Run Time 25 min.

4. Post time 5 min.

Results: As shown in Table 1, steam, ethylene oxide and gamma radiation sterilization of rifampin resulted in less recovery of rifampin than e-beam sterilization. After steam sterilization 19.5 to 34.3% of rifampin was recoverable from silicone sheet stock. After ethylene oxide sterilization of sheet stock incorporating minocycline and rifampin, only 46.0-74.1% of the incorporated rifampin was recovered. After gamma radiation, only 75.1-88.6% of the incorporated rifampin was recovered. In contrast, 92.4-95.1% of the incorporated rifampin was recovered after e-beam sterilization. High recovery was also seen with e-beam sterilization of silicon boots incorporating rifampin, with 90.2% of the rifampin being recovered after e-beam sterilization.

TABLE 1
Recoverable rifampin after sterilization

| Medium | | | | | |
|---|---|---|---|---|---|
| (polymer- | | | Post | Post | Post |
| thickness, | Pre-Sterilization | Post Steam | EtO | gamma | e-beam |
| geometry) | % | % | % | % | % |
| 4735–0.005″ | 100 | — | 46.0 | 81.4 | — |
| Sheet stock | | | | | |
| 4735–0.015″ | 100 | 34.3 | 72.0 | 82.8 | 95.1 |
| Sheet stock | | | 72.0 | 88.6 | 92.4 |
| 4850–0.005″ | 100 | 19.5 | 74.1 | 71.5 | — |
| Sheet Stock | | | 59.1 | | |
| 4850–0.015″ | 100 | 28.3 | 71.6 | 78.4 | — |
| Sheet Stock | | | | | |
| 4735–0.015″ | 100 | — | — | — | 90.2 |
| Boot | | | | | |
| 4850–0.005″ | 100 | — | 65.4 | — | — |
| Boot | | | | | |
| 4850–0.015″ | 100 | — | 77.5 | — | — |
| Boot | | | | | |

“—” = not tested

As shown in Table 2, minocycline withstood EtO sterilization and e-beam sterilization better than gamma radiation sterilization. After steam sterilization, no minocycline was recovered from silicone sheet stock. After ethylene oxide sterilization of sheet stock incorporating minocycline and rifampin, 86.8-100% of the incorporated minocycline was recovered. After gamma radiation, only 75.8-89.8% of the incorporated minocycline was recovered. 94.1-96.1% of the incorporated minocycline was recovered after e-beam sterilization. High recovery was also seen with e-beam sterilization of silicone boots incorporating minocycline, with 90.2% of the minocycline being recovered after e-beam sterilization.

Sterilization via steam, ethylene oxide, gamma radiation and e-beam was performed according to doses generally accepted by the device industry to be sterilizing for medical devices so that the level of sterilization achieved by each method was roughly equivalent. Accordingly, the results achieved appear to be rifampin-specific and minocycline-specific. Surprisingly, e-beam sterilization resulted in less degradation of rifampin than EtO and gamma radiation. E-beam sterilization also resulted in little degradation of minocycline.

TABLE 2
Recoverable minocycline remaining after sterilization

| Medium | | | | | |
|---|---|---|---|---|---|
| (polymer- | | | Post | Post | Post |
| thickness, | Pre-Sterilization | Post Steam | EtO | gamma | e-beam |
| geometry) | % | % | % | % | % |
| 4735–0.005″ | 100 | — | 86.8 | 86.1 | — |
| Sheet stock | | | | | |
| 4735–0.015″ | 100 | 0 | 100 | 84.2 | 96.1 |
| Sheet stock | | | 100.5 | 89.8 | 94.1 |
| 4850–0.005″ | 100 | 0 | 90.0 | 75.8 | — |
| Sheet Stock | | | 89.9 | | |
| 4850–0.015″ | 100 | 0 | 92.4 | 82.6 | — |
| Sheet Stock | | | | | |
| 4735–0.015″ | 100 | — | — | — | 90.2 |
| Boot | | | | | |
| 4850–0.005″ | 100 | — | 86.8 | — | — |
| Boot | | | | | |
| 4850–0.015″ | 100 | — | 99.6 | — | — |
| Boot | | | | | |

“—” = not tested

As can be seen by comparing the results presented in Tables 1 and 2, rifampin, but not minocycline, substantially degrades with EtO sterilization (46.0-77.5% remaining vs. 86.8-100% remaining, respectively). This is surprising in light of numerous reports indicating no degradation or loss of activity after EtO sterilization of rifampin-containing devices. This is further surprising in light of the numerous commercially available medical devices that are EtO sterilized and incorporate rifampin.

In addition, it is surprising that e-beam sterilization resulted in substantially greater recovery of both minocycline and rifampin than gamma radiation sterilization, as the level of sterilization was comparable.

What is claimed is:

1. A method for sterilizing a rifampin and minocycline-containing medical device, comprising:
sterilizing the device with e-beam radiation,
wherein (i) the amount of minocycline recoverable from the device after sterilization is 90% or more of the amount of minocycline recoverable from the device prior to sterilization, and (ii) the amount of rifampin recoverable from the device after sterilization is 90% or more of the amount of rifampin recoverable from the device prior to sterilization.

2. A method according to claim 1, wherein the amount of minocycline recoverable from the device after sterilization is 95% or more of the amount of minocycline recoverable from the device prior to sterilization.

3. A method according to claim 1, wherein the amount of rifampin recoverable from the device after sterilization is 95% or more of the amount of rifampin recoverable from the device prior to sterilization.

4. A method according to claim 1, wherein sterilizing the device with e-beam radiation comprises subjecting the device to about 25 to about 40 kGy of radiation.

5. A method according to claim 1, further comprising placing the device in packaging prior to sterilizing the device.

6. A method for manufacturing a medical device, comprising:
incorporating rifampin and minocycline in, on or about at least a portion of the medical device; and
sterilizing the at least a portion of the medical device with e-beam radiation,
wherein (i) the amount of minocycline recoverable from the device after sterilization is 90% or more of the amount of minocycline recoverable from the device prior to sterilization, and (ii) the amount of rifampin recoverable from the device after sterilization is 90% or more of the amount of rifampin recoverable from the device prior to sterilization.

7. A method according to claim 6, wherein the amount of minocycline recoverable from the device after sterilization is 95% or more of the amount of minocycline recoverable from the device prior to sterilization.

8. A method according to claim 6, wherein the amount of rifampin recoverable from the device after sterilization is 95% or more of the amount of rifampin recoverable from the device prior to sterilization.

9. A method according to claim 6, wherein sterilizing the device with e-beam radiation comprises subjecting the device to about 25 to about 40 kGy of radiation of radiation.

10. A method according to claim 6, further comprising placing the device in packaging prior to sterilizing the device.

11. A medical device sterilized by e-beam radiation, the device comprising a polymeric material incorporating minocycline and rifampin,
wherein (i) the amount of minocycline recoverable from the device after sterilization is 90% or more of the amount of minocycline recoverable from the device prior to sterilization, and (ii) the amount of rifampin recoverable from the device after sterilization is 90% or more of the amount of rifampin recoverable from the device prior to sterilization

12. A device according to claim 11, wherein the amount of minocycline recoverable from the device after sterilization is 95% or more of the amount of minocycline recoverable from the device prior to sterilization.

13. A device according to claim 11, wherein the amount of rifampin recoverable from the device after sterilization is 95% or more of the amount of rifampin recoverable from the device prior to sterilization.

14. A device according to claim 11, wherein sterilizing the device with e-beam radiation comprises subjecting the device to about 25 to about 40 kGy of radiation of radiation.

15. A device according to claim 11, wherein the polymeric material forms a catheter.

16. A device according to claim 11, wherein the polymeric material is disposed on or about a body member of the device.

17. A device according to claim 16, wherein the body member forms a housing of a neurostimulator, an infusion device, a defibrillator, a pacemaker, a stent, a catheter, or a lead.

18. A device according to claim 16, wherein the body member forms a housing of a neurostimulator.

19. A device according to claim 16, wherein the body member forms a housing of an infusion device.

20. A device according to claim 11, wherein the device is implantable.

21. A sterilized medical device comprising a polymeric material incorporating minocycline and rifampin, the device made by a process comprising:
incorporating rifampin and minocycline in, on or about at least a portion of the medical device; and
sterilizing the at least a portion of the medical device with e-beam radiation,
wherein (i) the amount of minocycline recoverable from the device after sterilization is 90% or more of the amount of minocycline recoverable from the device prior to sterilization, and (ii) the amount of rifampin recoverable from the device after sterilization is 90% or more of the amount of rifampin recoverable from the device prior to sterilization
